(19) United States
(12) Patent Application Publication
(10) Pub. No.: US 2009/0155341 A1
(43) Pub. Date: Jun. 18, 2009

(54) Medical device and a method for applying a biochemically active material on one or more body parts

(75) Inventors: Noam Gavriely, Haifa (IL); Oren Gavriely, Haifa (IL); Omer Kabak, Haifa (IL)

Correspondence Address: MARTIN D. MOYNIHAN d/b/a PRTSI, INC., P.O. BOX 16446, ARLINGTON, VA, 22215, US

(73) Assignee: OHK Medical Devices Ltd., Haifa (IL)

(21) Appl. No.: 12/314,501

(22) Filed: Dec. 11, 2008

Related U.S. Application Data
(60) Provisional application No. 61/006,033, filed on Dec. 14, 2007.

(51) Int. Cl. A61L 15/00

(52) U.S. Cl. 424/445

(57) ABSTRACT

A medical device having a sleeve-shaped elastic body which is rolled up to form an annular structure and may be rolled down to annularly contacting one or more body parts, such as limbs, a biochemically active material, such as a disinfecting material, added substantially to a portion of the sleeve-shaped elastic body, and a pressure-exerting element for rolling down the sleeve-shaped elastic body while applying an external pressure on a contact point between the portion and the one or more body parts.

### RELATED APPLICATIONS

This application claims the benefit of U.S. Provisional Patent Application No. 61/006,033 filed on Dec. 14, 2007, the contents of which are hereby incorporated by reference.

### FIELD AND BACKGROUND OF THE INVENTION

The present invention relates to an apparatus and a method for applying a biochemically active material on one or more body parts and, more particularly, but not exclusively to apparatus and a method for applying a biochemically active material on one or more body parts during, before, and/or after medical procedures, such as preoperative procedures.

Infections, such as surgical site infections (SSI) are a major source of prolonged illness and, less frequently, a cause of death in surgical patients.

For example, approximately 500,000 SSI occur each year and account for one fourth of the nosocomial infections in the United States. Many of these infections are minor in nature and may be treated on an outpatient basis. Major complications such as deep sternal infections have serious clinical consequences, including death.

The microbiology of SSI varies, depending on the type of surgery and procedure. According to the national nosocomial infections surveillance system, the most frequently isolated pathogens from SSI are staphylococcus aureus (20%) and coagulase-negative staphylococci (14%). These organisms are acquired from the exogenous environment or the patients' own skin flora. It should be noted that during the last years, there was an increase in the number of antibiotic-resistant bacterial strains.

In hospitals and medical centers, preoperative bathing or showering with skin antiseptics are practiced in order to prevent SSI. These procedures remove dirt, debris, and other residues and use any of a variety of antimicrobial formulations, such as Iodine/Iodophor, Alcohol, Parachlorometaxylenol, Triclosan, and Chlorhexidine Gluconate (CHG) as the active ingredient to reduce the microbial flora at the surgical site area. Usually, these procedures require stationary facilities and therefore cannot be efficiently performed before a field surgery.

Currently, the preoperative skin disinfection of a field surgery includes a process of applying a broad range of disinfecting solutions and a variety of antimicrobial formulations for disinfecting the patient's skin. The process includes using a brush or a sponge, such as a pre-impregnated sponge with a holder for spreading the solution on the patient's skin.

Another commonly performed pre-operative procedure, especially as part of a limb surgery, is stopping the blood flow to the surgical site area, usually using a tourniquet. Typically, such a procedure includes extracting the blood from the surgical site area and using a tourniquet for restraining a blood inflow from flowing toward the surgical site area.

During the last years, new varieties of devices have been developed for stopping the blood flow. For example, U.S Patent Application number 2005/0080450, published on Apr. 14, 2005, which is incorporated herein by reference, describes a silicone ring, wrapped with a tubular elastic sleeve and a set of pole straps. The silicone ring is designed to be placed to annularly contacting the distal end of the residual limb and the straps are pulled. After the silicone ring is positioned, it is rolled over the limb. The rolling applies a circumferential pressure on the limb, extracts the blood from the limb, and creates an arterial block that restrains the blood inflow from flowing toward the surgical site area.

### SUMMARY OF THE INVENTION

According to one aspect of the present invention there is provided a medical device for applying a biochemically active material on at least one body part of a patient. The method comprises a sleeve-shaped elastic body configured for being rolled up to form an annular structure and for being rolled down to annularly contacting at least one body part, a biochemically active material added substantially to a portion of the sleeve-shaped elastic body, and a pressure-exerting element configured for rolling down the sleeve-shaped elastic body while applying an external pressure on a contact point between the portion and the at least one body part.

Optionally, the pressure-exerting element comprises a tourniquet.

Optionally, the pressure-exerting element is a toroid-shaped element.

Optionally, the biochemically active material comprising a member of the group consisting of: a disinfecting material, an anesthetic, a desiccant, an antibiotic, an antineoplastic material, and an anti-inflammatory material.

Optionally, the biochemically active material is encapsulated in a plurality of capsules, the plurality of capsules being distributed in the portion.

Optionally, during the applying the pressure-exerting element is configured for applying an equally distributed external pressure on the portion, thereby displacing blood in the at least one body part toward the body of the patient.

Optionally, the pressure-exerting element is configured for extracting the biochemically active material toward the at least one body part during the applying.

Optionally, the sleeve comprising a member of the group consisting of: neoprene, cotton, spandex, polyurethane, polyester, vinyl, nylon, natural rubber, and any combination thereof.

Optionally, the medical device of claim 1 further comprises at least one additional biochemically active material added substantially to all of the portion.

Optionally, at least a segment of the sleeve-shaped elastic body is removable.

Optionally, the portion comprises a chemical detection material configured for reacting to the presence of the biochemically active material.

Optionally, the sleeve-shaped elastic body having a closed end lumen.

More optionally, the closed end lumen is a shaped as a comprising a member of the group consisting of: a glove, a sock, a fingerless glove, and a fingerless sock.

Optionally, the rolled down sleeve-shaped elastic body is configured for substantially decelerating the expansion of infectious agents on the surface of the at least one body part.

Optionally, the sleeve-shaped elastic body comprises a layer substantially impervious to fluids.

Optionally, the medical device of claim 1 further comprises an aperture for allowing the administering of a biochemically active material to the gap between the at least one body part and the sleeve-shaped elastic body.

More optionally, the aperture is adapted to be connected to a tube.

According to one aspect of the present invention there is provided a method for applying a biochemically active material on at least one body part. The method comprises a) adding a biochemically active material to at least a portion of a sleeve-shaped elastic body, b) rolling up the sleeve-shaped elastic body to form a hollow annular structure, and c) using a tubular pressure-exerting element for rolling down the sleeve-shaped elastic body to annularly contacting the at least one body part, thereby applying the biochemically active material on the skin of the at least one body part.

Optionally, the rolling down comprises applying an external pressure along the at least one body part.

Optionally, the adding comprises distributing substantially evenly capsules comprising the biochemically active material over the portion, further comprising tearing the capsules after the adding, thereby releasing the biochemically active material.

Optionally, the added biochemically active material is in an inactive form, further comprising activating the biochemically active material after the adding.

Optionally, the activating includes a member of the group consisting of: dissolving the biochemically active material, applying a physical force on the portion, radiating the portion with an ultrasonic energy, radiating the portion with an ultraviolet (UV) light waves, radiating the portion with an electromagnetic energy, changing the temperature of the portion.

Optionally, thereby substantially decelerating the expansion of infectious agents on the surface of the at least one body part after the rolling down.

According to one aspect of the present invention there is provided a medical device kit that comprises a sleeve-shaped elastic body configured for being rolled up to form an annular structure and for being rolled down to annularly contacting at least one body part and a container for containing a biochemically active material, the container being configured for applying the biochemically active material on a portion of the sleeve-shaped elastic body.

Optionally, the container is configured for spraying the biochemically active material on the sleeve-shaped elastic body.

According to one aspect of the present invention there is provided a medical device for applying a medical procedure on at least one body part on a patient, the medical device comprises a sleeve-shaped elastic body configured for being rolled up to form an annular structure and for being rolled down to annularly contacting at least one body part, the sleeve-shaped elastic body comprising a portion covered with a biochemically active material and a pressure-exerting element configured for rolling down the sleeve-shaped elastic body to annularly contacting the at least one body part, thereby simultaneously applying the biochemically active material on the at least one body part and displacing blood from the at least one body part toward the body of the patient.

Optionally, the pressure-exerting element is configured for clogging blood vessels between the at least one body part and the body of the patient.

According to one aspect of the present invention there is provided a preoperative medical device that comprises a cuttable sleeve-shaped elastic body configured for being rolled up to form an annular structure and for being rolled down to annularly contacting at least one body part and a biochemically active material covering substantially a portion of the sleeve-shaped elastic body.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1 and 2 are schematic illustrations of a medical device for applying a biochemically active material on a body part, such as a limb, according to one embodiment of the present invention;

FIG. 3 is a schematic illustration of an exemplary sock-shaped elastic body that is rolled down along a leg, according to one embodiment of the present invention;

FIG. 4 is a schematic illustration of the an exemplary sock-shaped elastic body which is socked with the biochemically active material and rolled down along a leg;

FIG. 5 is a schematic illustration of the medical device of FIG. 1 with a pair of handles in a rolled up condition, according to one embodiment of the present invention;

FIG. 6 is a schematic illustration of the medical device of FIG. 5 when it is applied on an exemplary limb, according to one embodiment of the present invention;

FIG. 7 is a flowchart of a method for applying a biochemically active material on one or more body parts and/or sections thereof, according to one embodiment of the present invention;

FIG. 8 is a schematic illustration of a medical device kit for applying a biochemically active material on one or more body parts or sections thereof; and

FIG. 9 is a schematic illustration of a brush that spreads a biochemically active material on the sleeve-shaped elastic body, according to one embodiment of the present invention.

### DESCRIPTION OF THE PREFERRED EMBODIMENTS

The present embodiments comprise a medical device and a method for applying a biochemically active material, such as a disinfecting material, on a body part, such as a limb, by rolling down a sleeve-shaped elastic body to annularly contacting at least a portion of the body part. The rolling down is performed using a pressure-exerting element, such as a toroid-shaped element. Optionally, the applying of the external pressure extracts the blood from the body part toward the rest of the body of the patient and substantially clogs blood vessels between the body part and the rest of the body. In such a manner, the medical device may be used for performing simultaneously two medical, optionally preoperative, procedures such as creating a bloodless area and applying the biochemically active material on the skin of the area that undergoes a medical treatment, such as a surgery.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The principles and operation of an apparatus and method according to the present invention may be better understood with reference to the drawings and accompanying description.

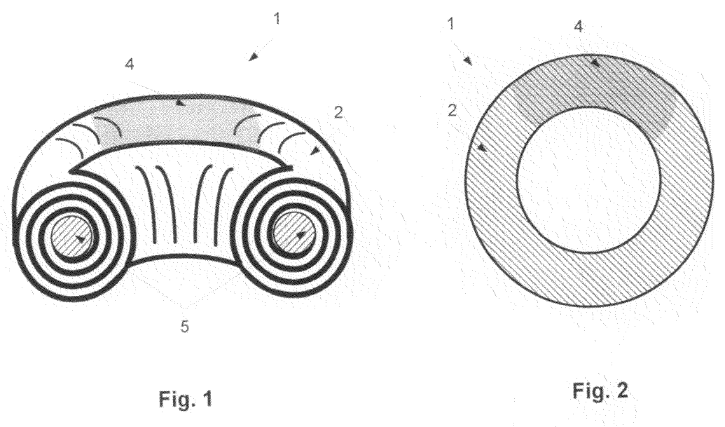

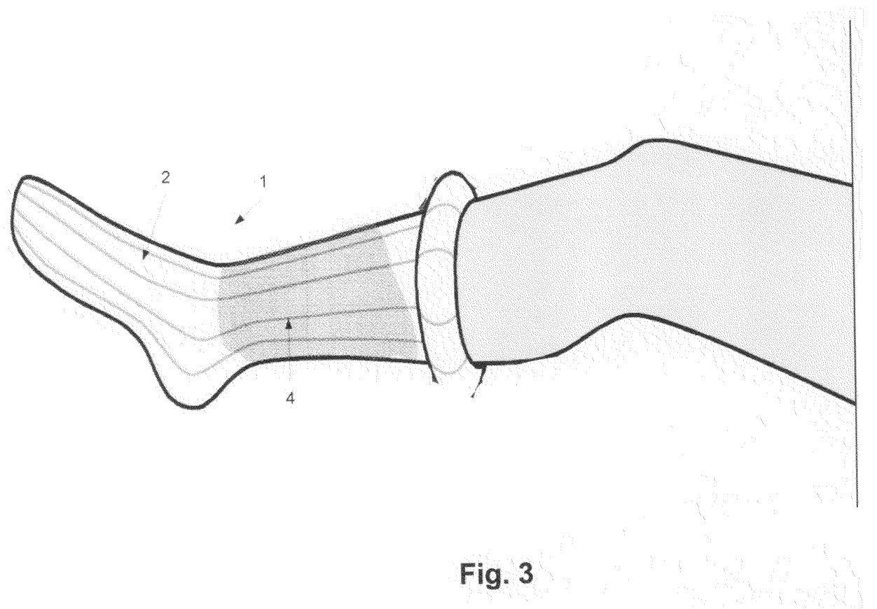

Reference is now made to FIGS. 1 and 2, which are schematic illustrations of a medical device 1 for applying a biochemically active material on a body part, such as a limb, according to one embodiment of the present invention. The medical device 1 comprises a sleeve-shaped elastic body 2 that is designed to be rolled up to form an annular structure, optionally as depicted in FIGS. 1 and 2 and for being rolled down to annularly contacting at least one body part, for example as depicted in FIG. 3, which is a schematic illustration of an exemplary sock-shaped elastic body 2 that is rolled down along a leg 3, as further described below. For clarity, the term annularly contacting should be understood as annularly contacting or substantially annularly contacting at least a portion of a body part, such as a limb. The sleeve-shaped elastic body 2 or at least a portion thereof 4 is covered with a biochemically active material 4. Optionally, the sleeve-shaped elastic body 2 is made of elastic knitted fabric, for example closely woven material cotton, such as a stockinette. The sleeve-shaped elastic body 2 may also be manufactured from any or a combination of neoprene, spandex, polyurethane, cotton, polyester, vinyl, nylon, and/or natural rubber.

The medical device 1 further comprises a pressure-exerting element 5, such as an elastic toroid-shaped element. The pressure-exerting element 5 is designed to roll down the sleeve-shaped elastic body 2 while exerting an external pressure on a contact point between the portion, which is covered with the biochemically active material 4, and the body part. Optionally, the pressure-exerting element 5 is made of a rubber tube, a strip of cloth, or any flexible material that can exert pressure on a body part, as described below. Optionally, the sleeve-shaped elastic body 2 is rolled around the pressure-exerting element 5 in a manner that forms a toroid-shaped element, as depicted in FIG. 1. Optionally, the pressure-exerting element 5 and the sleeve-shaped elastic body 2 are as described in U.S Patent Application number 2005/0080450, published on Apr. 14, 2005, which is incorporated herein by reference.

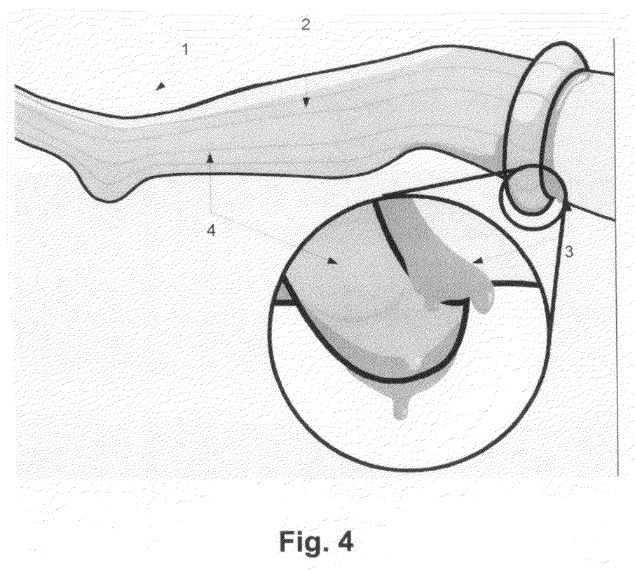

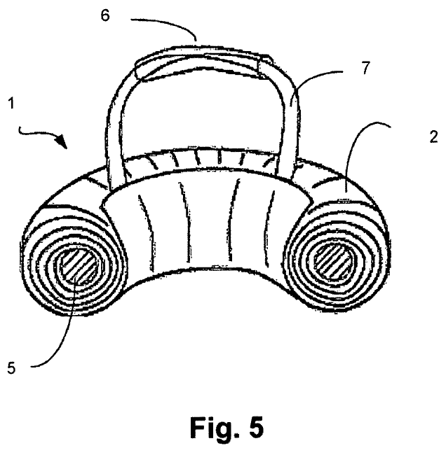

The portion of the biochemically active material 4 is absorbed into or adsorbed onto the sleeve-shaped elastic body 2. Optionally, the biochemically active material 4 is spread on a side of the sleeve-shaped elastic body 2 that is directed toward the body part when the sleeve-shaped elastic body 2 is roll down. Optionally, the sleeve-shaped elastic body 2 is substantially soaked with the biochemically active material 4. An example for such a substantially soaked sleeve-shaped elastic body 2 is shown at FIG. 4, which is a schematic illustration of the an exemplary sock-shaped elastic body 2 which is soaked with the biochemically active material 4 and rolled down along a leg 3.Optionally, the sleeve-shaped elastic body 2 is stored in or with a container that is substantially filled with the biochemically active material, optionally as described below.

Optionally, the sleeve-shaped elastic body 2 includes a layer, such as Stockinette or the like, which is impervious or semi-impervious to liquid. Optionally, the Stockinette or the like are an outer layer to the sleeve-shaped elastic body 2. This layer holds the biochemically active material securely in place and prevents drainage from seeping through. Optionally, this layer allows applying of the biochemically active material on the limb, while preventing from fluids, such as water, to be absorbed by the sleeve-shaped elastic body 2.

Optionally, the sleeve-shaped elastic body 2 is coated or substantially coated with an outer and/or an inner layer of 3M™ Steri-Drape™ incise drape that assists in preventing viral contamination, and bacterial infection during and after the surgical procedure.

The medical device may used for covering the body part with any component or compound that is required or desired for a certain medical procedure such as a surgery. Optionally, the biochemically active material 4 includes a disinfecting material, such as an alcohol-based material, quaternary ammonium, or other cleansing agent or compound, an anesthetic, such as lidocaine, cortisone like drug, such as triamcinolone, a desiccant, such as alumina or silica gel, an antineoplastic material, such as methotrexate (MTX) and an anti-inflammatory material, such as Ibuprofen or Naprosyn. and/or an antibiotic, such as penicillin. In one embodiment, the biochemically active material 4 includes a disinfecting material, optionally an Iodine-based disinfecting material such as Betadyne, Hibaclense, Chlorhexodine, or a germicide, which may be referred to as an antiseptic material, such as Thymol, Betadine, or an alcohol gel Sanitizer.

Optionally, the biochemically active material 4 is a liquid or gel medication that is impregnated in and/or layered the sleeve-shaped elastic body 2 during the manufacturing process. Optionally, the biochemically active material 4 is a powder or a granular medication that is evenly spread on the aforementioned portion of the sleeve-shaped elastic body. Optionally, the biochemically active material 4 is encapsulated in small containers such as micro or mini capsules, which are distributed substantially evenly on and/or in the aforementioned portion of the sleeve-shaped elastic body 2.

In an alternative embodiment, the biochemically active material 4 is in inactive form needs to be activated, for example by dipping the sleeve-shaped elastic body 2 or a portion thereof in an activation solution such as water, alcohol or any other chemical solution and/or solvent. In one embodiment, the biochemically active material 4 is optionally in powder or granules form.

As described the biochemically active material 4 may be encapsulated in containers such as in micro or mini capsules. In order to release the biochemically active material 4, the capsules have to be torn. The tearing of the capsules may be facilitated by dissolving the capsules walls with a solvent such as water, applying a physical force on the sleeve-shaped elastic body 2 that carries the capsules, and/or radiating the sleeve-shaped elastic body 2 with a certain energy, such as an ultrasonic energy, a ultraviolet (UV) light waves, or an electromagnetic energy. Optionally, the bioactive material may be released by changing the temperature of the portion of the sleeve-shaped elastic body 2 that carries the capsules. Optionally, the capsules are torn by the pressure which is extracted by the pressure-exerting element 5 during the rolling down of the sleeve-shaped elastic body 2. As described above, the pressure-exerting element 5 extracts pressure on one or more contact points between the portion, which is covered with the biochemically active material 4, and the body part 3. This pressure tears the capsules and releases the biochemically active material which has been encapsulated therein. It should be noted that the tearing may be performed before, during, and/or after the placement of the sleeve-shaped elastic body 2 on the body part, for example as depicted in FIG. 3. Optionally, the capsules are contain ChloraPrep™ that comprises 2 percent chlorhexidine gluconate (CHG) and 70 percent isopropyl alcohol (IPA) patient preoperative skin preparation.

As described above, when the pressure-exerting element 5 is used to roll down the sleeve-shaped elastic body 2, it applies an external pressure on the contact points between the portion, which is covered with the biochemically active material 4, and the body part 3. In one embodiment of the present invention, the pressure-exerting element 5 is used as a tourniquet. When the pressure-exerting element 5 is placed to annularly contacting at least a portion of a body part, such as a limb, it stops the flow of blood from one or more blood vessels of the body part by exerting external pressure, as described above. The external pressure is applied circumferentially upon the skin and underlying tissues of the limb. This external pressure is transferred to the walls of vessels, causing them to become temporarily occluded.

It should be noted that the medical device may be used for performing medical, optionally preoperative, procedures, both in humans and animals.

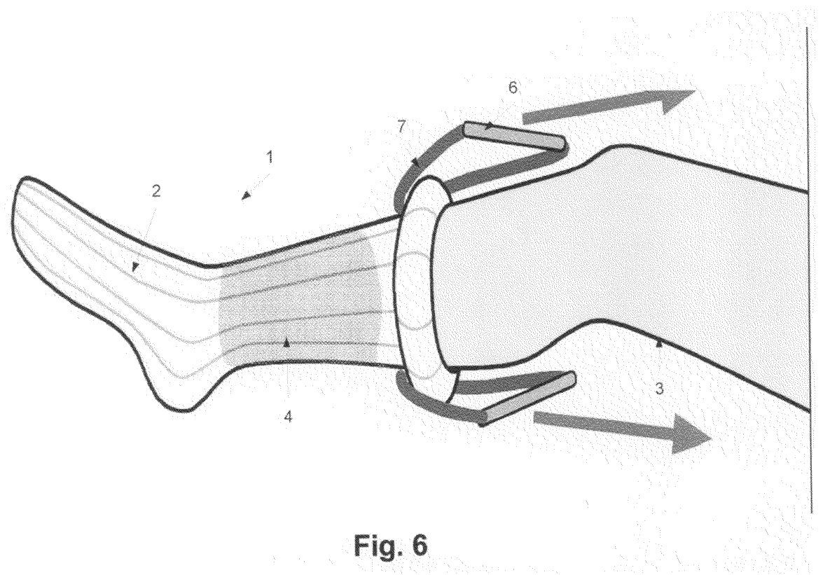

Reference is now also made to FIGS. 5 and 6. FIG. 5 is a schematic illustration of the medical device 1 with a pair of handles 6 in a rolled up condition, according to one embodiment of the present invention. FIG. 6 is a schematic illustration of the medical device 1 with the pair of handles 6, when it is applied on an exemplary limb, a human leg, according to one embodiment of the present invention. The sleeve-shaped elastic body 2 is as depicted in FIGS. 1 and 2, however in FIG. 6 the medical device 1 further includes the handles 6, each connected to a strip 7 which is rolled up with the sleeve-shaped elastic body 2, for example as depicted in 5. In such an embodiment, the pulling of the handles 6 rolls down the sleeve-shaped elastic body 2 of the medical device 1 along the limb and exerts the aforementioned external pressure on the body part 3, as described above.

Optionally, the medical device 1 is used as a preoperative tool for preparing a body part 3, such as a limb, for a surgery, optionally a field surgery, by reducing the amount of blood in the limb and clogging blood vessels between the limb 3 and/or an area thereof and the rest of the body of the patient. In particular, the rolling of the pressure-exerting element 5 on the top of the limb 3 substantially displaces the blood from the blood vessels of the limb 3 toward the body of the patient and optionally clogs blood vessels between the limb 3 and/or an area thereof, allowing a medical practitioner to work in a bloodless operative field. In such an embodiment, the amount of blood that could have been lost during the surgery is reduced. The blood that is displaced toward the body of the patient is not lost during the surgery as has been extracted from the area that undergoes a surgery.

Optionally, at least a portion of the sleeve-shaped elastic body 2 is cuttable or includes openable segments. Such openable segments may include zipper-like edges, scotch bright pads, and/or temporary seams which can easily be unstitched. Such portions allows medical practitioner, such as a physician, a nursing person, and/or a paramedic, to easily access an area of the body part 3 that is covered by the sleeve-shaped elastic body 2 without removing the sleeve-shaped elastic body 2 and/or the pressure-exerting element 5, which is optionally used as a tourniquet, from the body part 3. Such an openable segment may be designed to allow operations such as knee operations, release of carpel tunnel operations and the like. Such an adapted openable segment may be designed according to a procedure of a specific operation.

Optionally, the openable segment is covered with a layer of 3M™ Steri-Drape™ incise drape that assists in preventing viral contamination, and bacterial infection during and after the surgical procedure.

Optionally, the sleeve-shaped elastic body 2 includes a portion 4 having a number of layers of one or more biochemically active materials. In such a manner, the sleeve-shaped elastic body 2 may release a number of biochemically active materials in a sequential, a simultaneous, and/or a scheduled manner. Optionally, the order of the layered biochemically active materials is determined according to the requirements of different stages of a certain medical procedure.

Optionally, the portion 4 includes a detection material that reacts to the presence and/or the activity of the biochemically active material. In such a manner, the user of the medical device 1 receives an indication that the biochemically active material is present and optionally active.

The sleeve-shaped elastic body 2, which is depicted in FIGS. 3 and 6, has a closed end lumen that gives the sleeve-shaped elastic body 2 a sock shape. The sleeve-shaped elastic body 2 may also have an open end lumen, a semi open end lumen having a fingerless glove end or fingerless sock shape end, or any other end which is adapted to a certain body part and/or can support the stretching of the sleeve-shaped elastic body 2 during the rolling down thereof along the body part 3.

As described above, the sleeve-shaped elastic body 2 is designed to be applied on a body part or a portion thereof when it is rolled down. The applied sleeve-shaped elastic body 2 covers the body part or the portion thereof. Such coverage substantially decelerates the expansion of infectious agents on the surface of the covered body part. In particular, an infection or a concentration infectious agents, such as contaminates and/or toxins, which is located in a proximity to the body part may mitigate toward an open wound or an area that undergoes a surgery. By covering the open wound and/or the area that undergoes a surgery and/or by covering the infection or the aforementioned concentration with the sleeve-shaped elastic body 2, the user substantially decelerates the expansion of the infectious agents or the infection toward the open wound and/or the area that undergoes a surgery. The sleeve-shaped elastic body 2 physically decelerates the aforementioned migration as it absorbs the infection or the concentration. Optionally, the sleeve-shaped elastic body 2 includes a biochemically active material, such as a disinfecting material, that destroys the microorganisms or the pathogens which may be in the infection or the concentration by cleansing.

Optionally, the sleeve-shaped elastic body 2 includes an aperture, optionally closable, which connected and/or adapted to be connected to tube. The aperture allows the administrating of pharmaceutical materials, such as the aforementioned biochemically active materials, into the gap between the sleeve-shaped elastic body 2 and covered limbed that optionally has been sterilized, as described above. Optionally, the aperture is positioned in the closed end lumen of the sleeve-shaped elastic body 2, for example in one of the fingertip ends.

Optionally, the aperture is connected or adapted to be connected to a dispenser that is designed to release one or more biochemically active materials at a measured rate. Optionally, the dispenser comprises a biochemically active material storage and a reclosable port formed to permit release of biochemically active material at a predetermined variable controlled rate. Such a port permits initiation, termination of the biochemically active materials dispensing as well as acceleration and deceleration of the rate of dispensing through port. Optionally, the dispenser is designed to deliver medical fluids via the aforementioned aperture at controlled delivery or infusion rates. Optionally, the dispenser comprises a peristaltic pump that engages a resilient tube that is coupled between the biochemically active material storage and the port. The biochemically active material storage may include an intravenous (IV) bag, a syringe, and the like. The peristaltic pump delivers the medical fluid to the patient by sequentially squeezing and releasing sections of the tube in a wavelike pattern. Another type of pump is the syringe pump, which pumps medical fluid from a syringe to a patient by pushing down on the plunger of the syringe at a controlled rate.

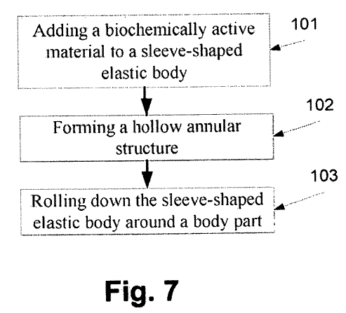

Reference is now made to FIG. 7, which is a flowchart of a method for applying a biochemically active material on one or more body parts or sections thereof, according to one embodiment of the present invention. As shown at 101, the method is based on adding, for example by impregnating and/or coating, a biochemically active material, for example as described above, to at least a portion of a sleeve-shaped elastic body. Then, as shown at 102, the sleeve-shaped body with added biochemically active material is rolled to form a hollow annular structure, for example as depicted in FIG. 1. Now, as shown at 103, a tubular pressure-exerting element is used for rolling down the sleeve-shaped elastic body to annularly contacting the one or more body parts. The rolling down applies the biochemically active material on the skin of the body parts. Optionally, the rolling down also applies an external pressure along the body parts. As described above, such an external pressure displaces blood from the body parts toward the body of the patient and clogs blood vessels between the body part and/or an area thereof and the rest of the body of the patient. In such a manner, a medical practitioner may follow 101-103 to perform simultaneously two medical, optionally preoperative, procedures, for example removing blood from the body parts and applying a biochemically active material, such as a disinfecting material, on the skin of the body part.

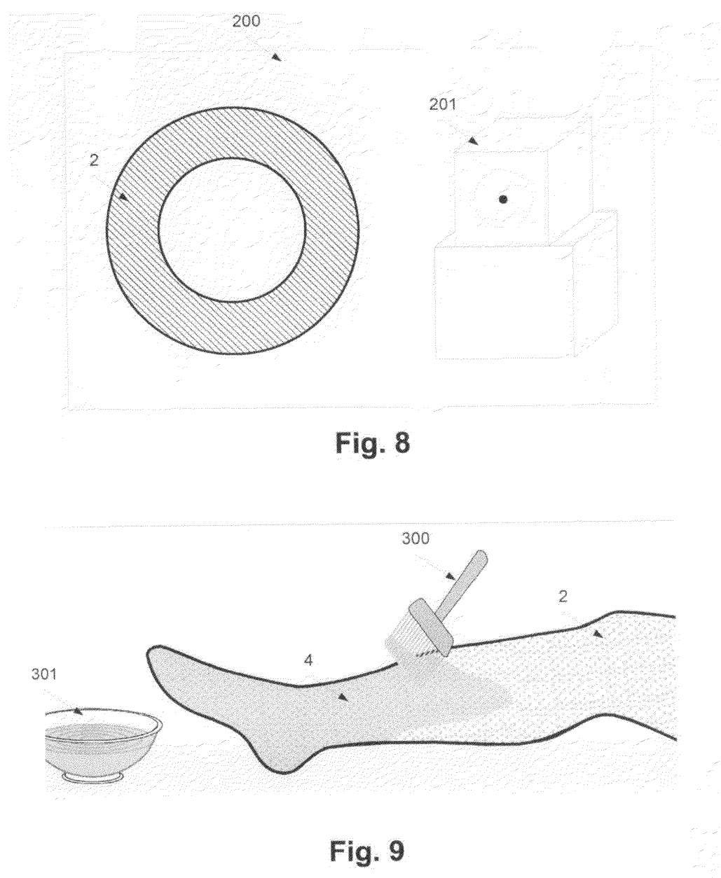

Reference is now made to FIG. 8, which is a schematic illustration of a medical device kit 200 for applying a biochemically active material on one or more body parts or section thereof, according to one embodiment of the present invention. The kit 200 includes the sleeve-shaped elastic body 2 in a rolled up form, as depicted in FIG. 1, and a container 201 for containing a biochemically active material. Optionally, the container is a sprayer, a rolling pan, or any other apparatus that may be used for both containing the biochemically active material and applying it on a portion of the sleeve-shaped elastic body. Optionally, the container 201 is a regular can or bottle. In such an embodiment, the kit 200 further comprises a brush, a sponge, or another apparatus which can be used for spreading the biochemically active material on the sleeve-shaped elastic body 2, for example as shown at FIG. 9. A kit 200 that comprises such a brush 300 and a can 301 may be used for adding the biochemically active material to a portion 4 of the sleeve-shaped elastic body 2 when it is in a rolled down form.

It is expected that during the life of this patent many relevant devices and systems will be developed and the scope of the terms herein, particularly of the terms a sleeve-shaped elastic body and a biochemically active material are intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A medical device for applying a biochemically active material on at least one body part of a patient, comprising:
a sleeve-shaped elastic body configured for being rolled up to form an annular structure and for being rolled down to annularly contacting at least one body part;
a biochemically active material added substantially to a portion of said sleeve-shaped elastic body; and
a pressure-exerting element configured for rolling down said sleeve-shaped elastic body while applying an external pressure on a contact point between said portion and said at least one body part.

2. The medical device of claim 1, wherein said pressure-exerting element comprises a tourniquet.

3. The medical device of claim 1, wherein said pressure-exerting element is a toroid-shaped element.

4. The medical device of claim 1, wherein said biochemically active material comprising a member of the group consisting of: a disinfecting material, an anesthetic, a desiccant, an antibiotic, an antineoplastic material, and an anti-inflammatory material.

5. The medical device of claim 1, wherein said biochemically active material is encapsulated in a plurality of capsules, said plurality of capsules being distributed in said portion.

6. The medical device of claim 1, wherein during said applying said pressure-exerting element is configured for applying an equally distributed external pressure on said portion, thereby displacing blood in said at least one body part toward the body of the patient.

7. The medical device of claim 1, wherein said pressure-exerting element is configured for extracting said biochemically active material toward said at least one body part during said applying.

8. The medical device of claim 1, wherein said sleeve comprising a member of the group consisting of: neoprene, cotton, spandex, polyurethane, polyester, vinyl, nylon, natural rubber, and any combination thereof.

9. The medical device of claim 1, further comprising at least one additional biochemically active material added substantially to all of said portion.

10. The medical device of claim 1, wherein at least a segment of said sleeve-shaped elastic body is removable.

11. The medical device of claim 1, wherein said portion comprises a chemical detection material configured for reacting to the presence of said biochemically active material.

12. The medical device of claim 1, wherein said sleeve-shaped elastic body having a closed end lumen.

13. The medical device of claim 12, wherein said closed end lumen is a shaped as a comprising a member of the group consisting of: a glove, a sock, a fingerless glove, and a fingerless sock.

14. The medical device of claim 1, wherein said rolled down sleeve-shaped elastic body is configured for substantially decelerating the expansion of infectious agents on the surface of said at least one body part.

15. The medical device of claim 1, wherein said sleeve-shaped elastic body comprises a layer substantially impervious to fluids.

16. The medical device of claim 1, further comprising an aperture for allowing the administering of a biochemically active material to the gap between said at least one body part and said sleeve-shaped elastic body.

17. The medical device of claim 16, wherein said aperture is adapted to be connected to a tube.

18. A method for applying a biochemically active material on at least one body part, comprising:
a) adding a biochemically active material to at least a portion of a sleeve-shaped elastic body;
b) rolling up said sleeve-shaped elastic body to form a hollow annular structure; and
c) using a tubular pressure-exerting element for rolling down said sleeve-shaped elastic body to annularly contacting the at least one body part, thereby applying said biochemically active material on the skin of the at least one body part.

19. The method of claim 18, wherein said rolling down comprises applying an external pressure along said at least one body part.

20. The method of claim 18, wherein said adding comprises distributing substantially evenly capsules comprising said biochemically active material over said portion, further comprising tearing said capsules after said adding, thereby releasing said biochemically active material.

21. The method of claim 18, wherein said added biochemically active material is in an inactive form, further comprising activating said biochemically active material after said adding.

22. The method of claim 21, wherein said activating includes a member of the group consisting of: dissolving said biochemically active material, applying a physical force on said portion, radiating said portion with an ultrasonic energy, radiating said portion with an ultraviolet (UV) light waves, radiating said portion with an electromagnetic energy, changing the temperature of said portion.

23. The method of claim 18, thereby substantially decelerating the expansion of infectious agents on the surface of the at least one body part after said rolling down.

24. A medical device kit, comprising:
a sleeve-shaped elastic body configured for being rolled up to form an annular structure and for being rolled down to annularly contacting at least one body part; and
a container for containing a biochemically active material, said container being configured for applying said biochemically active material on a portion of said sleeve-shaped elastic body.

25. The medical device of claim 2,4, wherein said container is configured for spraying said biochemically active material on said sleeve-shaped elastic body.

26. A medical device for applying a medical procedure on at least one body part on a patient, said medical device comprising:
a sleeve-shaped elastic body configured for being rolled up to form an annular structure and for being rolled down to annularly contacting at least one body part, said sleeve-shaped elastic body comprising a portion covered with a biochemically active material; and
a pressure-exerting element configured for rolling down said sleeve-shaped elastic body to annularly contacting the at least one body part, thereby simultaneously applying said biochemically active material on the at least one body part and displacing blood from the at least one body part toward the body of the patient.

27. The medical device of claim 26, wherein said pressure-exerting element is configured for clogging blood vessels between the at least one body part and the body of the patient.

28. A preoperative medical device, comprising:
a cuttable sleeve-shaped elastic body configured for being rolled up to form an annular structure and for being rolled down to annularly contacting at least one body part; and
a biochemically active material covering substantially a portion of said sleeve-shaped elastic body.
